(19) United States
(12) Patent Application Publication
(10) Pub. No.: US 2010/0068213 A1
(43) Pub. Date: Mar. 18, 2010

(54) SELECTED COLOSTRUM FOR TREATMENT OF INTESTINAL BARRIER FUNCTION DISORDERS

(75) Inventor: Houkje Bouritius, Zeist (NL)

Correspondence Address: FOLEY AND LARDNER LLP;SUITE 500, 3000 K STREET NW, WASHINGTON, DC, 20007, US

(73) Assignee: N.V. NUTRICIA, Zoetermeer (NL)

(21) Appl. No.: 12/527,772

(22) PCT Filed: Feb. 20, 2007
(86) PCT No.: PCT/NL2007/050071

§ 371 Date: Aug. 19, 2009

(51) Int. Cl. A61K 39/395; A61P 1/00

(52) U.S. Cl. 424/157.1

(57) ABSTRACT

The invention relates to the treatment of intestinal barrier function disorders involving administration of a selected colostrum comprising more than 25 weight % undenatured immunoglobulin G(IgG) based on the weight of total protein content of said colostrum to a subject. The invention further relates to compositions comprising colostrum and indigestible dietary fibers wherein the colostrum comprises more than 25 weight % undenatured immunoglobulin G based on the weight of total protein content of said colostrum.

### FIELD OF THE INVENTION

The invention relates to the treatment of intestinal barrier function disorders involving administration of colostrum. The invention further concerns compositions comprising colostrum.

### BACKGROUND OF THE INVENTION

WO 03041512 discloses compositions comprising colostrum in combination with pro- and prebiotics beneficial for the gastrointestinal health of an animal.

WO 2004041004 discloses compositions comprising colostrum wherein the bioactive bovine colostrum components should comprise 10-40 wt % bovine colostrum powder of 20-25 wt % IgG1 on dry weight basis and may further comprise plant polysaccharides and low molecular weight carbohydrates. The IgG1 is described to have a function in passively protecting against gastro-intestinal infections by various enteric pathogens, thereby preventing pathogen induced diarrhea.

In the prior art no selection has been made of colostrums that are specifically beneficial for the treatment of intestinal barrier function disorders which for example has the disadvantage that potentially ineffective sources of colostrum are used.

### SUMMARY OF THE INVENTION

It is an object of the invention to provide optimal use of colostrum for the treatment of intestinal barrier dysfunction.

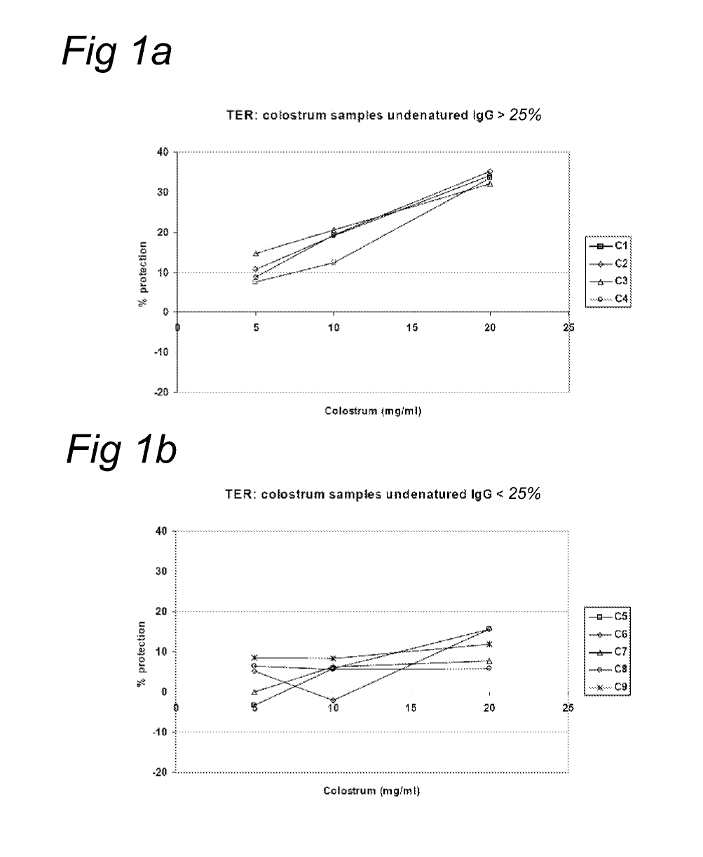

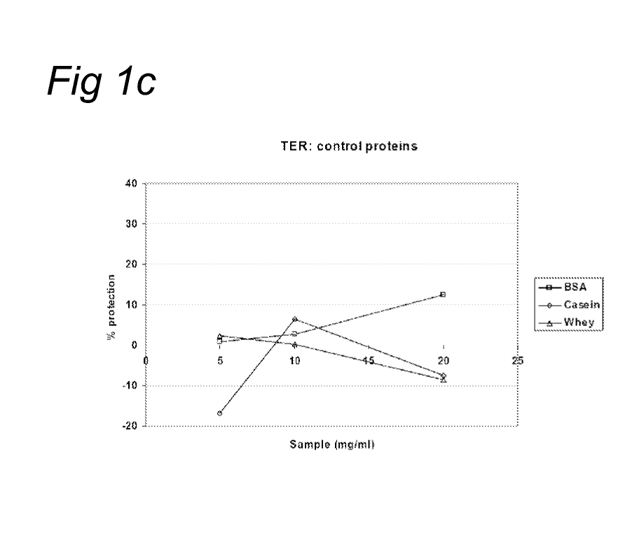

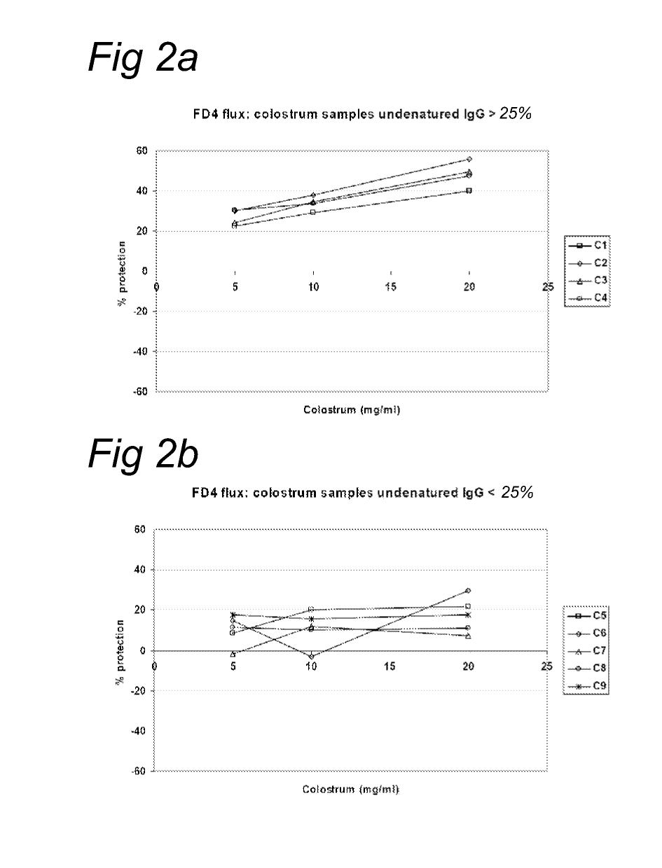

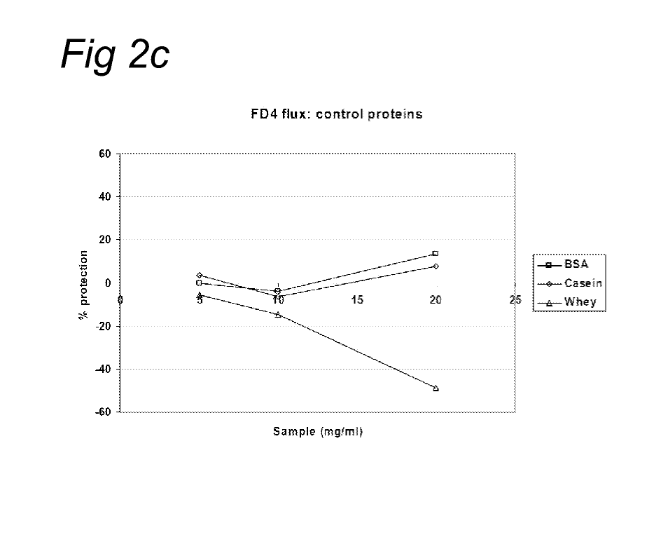

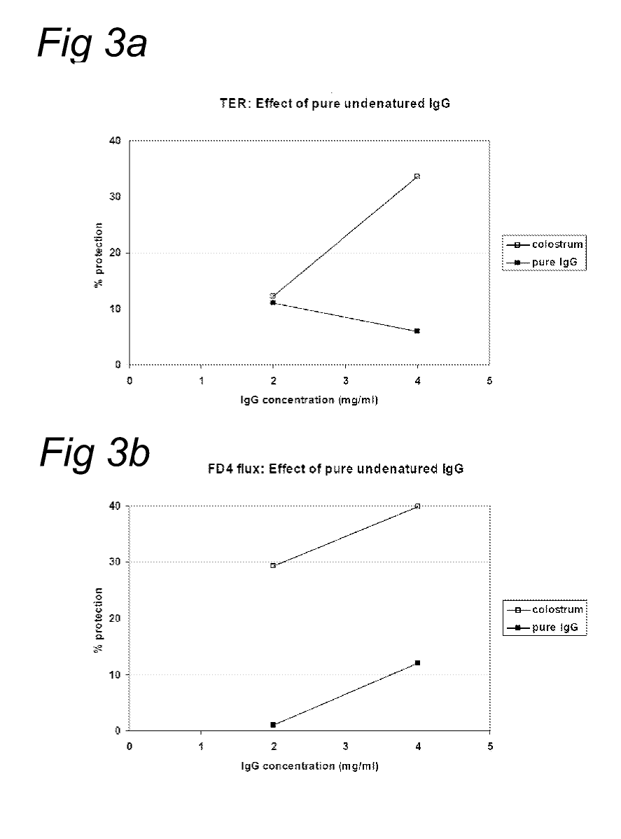

The inventors surprisingly found that not all colostrums are suitable for the treatment of intestinal barrier function disorders. In a model for intestinal barrier function it has been found that certain colostrum samples have a significant effect on the barrier function while others do not have this effect. This can be seen from FIGS. 1 and 2 from the Examples section hereinbelow. After further investigations it was found that there was a correlation with the presence of undenatured immunoglobulin G (IgG) in the colostrum samples. When the colostrum contained more than 25 weight percent of undenatured IgG based on the total weight of the colostrum protein, there was a significant positive effect on barrier function as can be seen in table 1 and FIGS. 1 and 2 of the Examples section hereinbelow. It is not the IgG itself that is causing the effect, since pure, undenaturated, IgG does not have this effect, see FIG. 3 of the Examples section hereinbelow.

To the best of our knowledge, up to date it has not been suggested that for the treatment of intestinal barrier function disorders one should select the colostrum on the basis of a minimum content of undenatured IgG.

Thus according to the invention the colostrum that is used for the treatment of intestinal disorders should comprise more than 25 weight % undenatured immunoglobulin G based on the weight of total protein in colostrum powder.

### DETAILED DESCRIPTION OF THE INVENTION

The invention concerns a method for the therapy of intestinal disorders, said method comprising the administration of colostrum comprising more than 25 weight % undenatured immunoglobulin G (IgG) based on the weight of total protein content of said colostrum to a subject.

For certain jurisdictions the invention can be worded as the use of colostrum for the manufacture of a medicament for the treatment and/or prevention of intestinal disorders wherein the colostrum comprises more than 25 weight % undenatured immunoglobulin G (IgG) based on the weight of total protein content of said colostrum. Alternatively the present invention can be worded as a composition comprising colostrum comprising more than 25 weight % undenatured immunoglobulin G (IgG) based on the weight of total protein content of said colostrum for the treatment and/or prevention of intestinal disorders.

Preferably the colostrum comprises between 25-60 wt. % undenatured IgG and more preferably the colostrum comprises between 25-40 wt. % undenatured IgG, which is the most economically feasible product.

The invention further relates to compositions comprising colostrum and indigestible dietary fibers wherein the colostrum comprises more than 25 weight % undenatured immunoglobulin G based on the weight of total protein content of said colostrum.

In the context of this invention ‘total protein content of colostrum’ includes denatured and undenatured immunoglobulin G.

### Intestinal Barrier Function

In the context of this invention the intestinal barrier is regarded as the ability of the intestine to maintain selectivity to its external environment. The role of the barrier is preventing the entrance into the body of potentially harmful antigenic, toxic, or carcinogenic compounds.

There are numerous components that together form the barrier function. These include the surface mucus layer, the absorptive brush border cell membrane of the enterocytes, the paracellular junctional areas, the epithelial and sub-epithelial immune defence mechanisms and the intestinal lymph nodes.

Disruption of the intestinal barrier may play an important role in the etiology and pathogenesis of various intestinal and systemic diseases. Under certain pathophysiologic conditions gut barrier function may be impaired to such extent that this can lead to the escape of bacteria or their products (endotoxin) which has been termed ‘bacterial translocation’. Bacterial translocation may thus be defined as the movement of bacteria or bacterial products across the intestinal membrane for example to emerge in the lymphatics or the visceral circulation. Furthermore, failure of the integrity of the intestinal epithelium permits absorption of lipopolysaccharide and other toxins, which can cause both local and systemic diseases. As a consequence, the patient can lose lean body mass and/or becomes nutritionally depleted.

In the context of this invention the term ‘intestinal disorders’ more specifically concerns intestinal barrier related disorders. The term ‘intestinal barrier related disorder’ in the context of the present invention relates to diseases wherein intestinal barrier function is impaired.

Different diseases are associated with increased intestinal permeability. Also certain drugs and alcohol abuse have been reported to increase intestinal permeability. In an embodiment compositions according to the present invention are used for the therapy of diseases and conditions selected from the group consisting of inflammatory bowel disease (e.g. Crohn's disease, ulcerative colitis), HIV infection/AIDS, food allergy, non-steroidal anti-inflammatory drugs induced intestinal disorder (e.g. ulcerations), disorders resulting from fasting, disorders resulting from total parenteral nutrition, disorders resulting from abdominal surgery and renal disease. In one embodiment the present invention concerns the treatment of diseases associated with increased intestinal permeability, in particular the diseases and disorders as mentioned above. In one embodiment the present invention concerns the prevention of diseases associated with increased intestinal permeability, in particular the diseases and disorders as mentioned above. In one embodiment the present invention relates to the treatment and/or prevention of intestinal disorders in HIV patients.

### Impaired Gut Health in HIV

Various clinical manifestations of gut dysfunction in HIV patients have been described, including (chronic) diarrhea, malabsorption, weight loss, (acute) infection sometimes followed by bacteraemia, hemorrhagic colitis, and abdominal pain. The majority of HIV infected individuals experience an increased burden of these gastrointestinal symptoms during the progression of disease. Pathophysiological changes underlying these manifestations include increased epithelial permeability and impaired enterocyte function. Permeability is usually assessed by measuring the permeation of sugars across the mucosa and detecting the recovery in the urine.

These manifestations of gut dysfunction, including carbohydrate and lipid malabsorbtion, and increased small bowel transepithelial permeability, can often be detected in HIV infected individuals, not undergoing highly active antiretroviral therapy (HAART) yet, even without obvious manifestations of clinical symptoms, but with a progressive reduction of CD4 counts. Indeed, these aspects of gut dysfunction are more prevalent and become more severe in AIDS patients with symptoms such as diarrhea. In addition, there is collective evidence that there also is progressive increased intestinal permeability. The incidence can range from 20-25% of HIV asymptomatic individuals up to 100% in AIDS patients with diarrhea.

In-vivo permeability can conveniently be assessed by measuring the permeation of sugars such as lactulose and rhamnose across the mucosa and detecting the recovery in the urine. In a number of studies using different markers, like D-xylose, mannitol and lactulose, as part of a sugar absorption/permeability tests, abnormal small intestinal absorption was demonstrated in HIV patients without clinical symptoms. Although the incidence of impaired marker uptake was greater in the symptomatic HIV groups and even more pronounced in AIDS patients, these abnormal results suggest that already in HIV asymptomatic individuals, intestinal enterocyte function may be impaired [1].

Increased epithelial permeability and/or impaired enterocyte function may contribute to undernourishment in HIV patients. Ultimately a variety of manifestations may develop as a result of inadequate nutrient uptake such as accelerated disease progression, reduced immune function, increased health care utilization, and lowered survival rate.

Thus in one embodiment the present invention relates to the treatment and/or prevention of malabsorption in HIV patients.

HIV infected patients with gastrointestinal symptoms show low grade small bowel atrophy and a maturational defect in enterocytes. This can result in defective brush border assembly and differentiation which causes malabsorbtion and increased secretion into the bowel lumen, resulting in diarrhea [2].

The etiology of HIV associated intestinal dysfunction is largely unknown, and has been variously attributed to opportunistic infections and cytokine secretion in response to inflammation. It is uncertain if a mucosal inflammatory response in the intestine is a result of HIV infection or altered enterocytes function and activity. It is clear, however, that during the cause of disease there is a distinct pattern of local pro-inflammatory cytokine production [3-7]. Especially TNF-α, IL-β and IL-6 and to a lesser extent IFN-γ are found to be elevated in intestinal biopsies of HIV infected patients. Inflammation can produce a leaky gut, an immune compromised individual does not need to ingest a foreign organism to have inflammatory diarrhea, they will get diarrhea because the normal balance of intestinal flora and other elements of the nonspecific immune defense system is altered, allowing antigens to cross the leaky gut.

It has been shown in models of intestinal barrier function that pro-inflammatory cytokines can have strong detrimental effects on intestinal disruption by increasing paracellular permeability [8-10]. Indeed, isolated immune cells from HIV patients release vast amounts of these proinflammatory cytokines that are involved in the increased epithelial permeability. In-vitro epithelial barrier function of the duodenal mucosa of HIV infected patients was characterized by epithelial resistance and by lactulose/mannitol flux measurements and this resulted in decreased epithelial resistance from HIV infected patients with diarrhea and a concomitantly increased mucosal-to-serosal lactulose flux [11]. A decrease in transepithelial resistance results in an increase of paracellular permeability caused by the disruption of the tight junctions. The in-vitro models used in the examples mimic this inflammatory gut effect in the HIV infected patient by using a cytokine mixture. This too causes a decrease in transepithelial resistance (TER), see FIGS. 1 and 3a, in an intestinal cell line monolayer cultured on a transwell insert and cause an increase of the mucosal-to-serosal flux of a FITC-dextran molecule (FD4), see FIGS. 2 and 3b.

### Colostrum

Colostrum is a pre-milk fluid secreted directly after birth. In cows, it is produced in excess and only partly consumed by the calf. The remaining portion can then be collected making colostrum a natural food ingredient. Bovine colostrum comprises a mixture of proteins, fat and sugars. The protein part contains among other normal milk proteins large amounts of immunoglobulins and other serum proteins that are presumed to have an effect on the newborn. The composition of colostrum is quite different from that of ruminant milk in established lactation.

Colostrum according to this invention is defined as milk from the first four milkings collected during the first 48 hours after parturition or even the first milkings collected during the first 24 hours after parturition. If the collection of colostrum does not take place shortly after birth, the bioactive components in colostrum will rapidly deteriorate with time. Whole colostrum can be fractionated and utilized as separate components.

The protein content of bovine colostrum is three to four times higher—up to 150 grams per litre compared to 30 to 40 grams per litre—than it is in regular cow's milk. The greater part of this protein is comprised of whey proteins. Immunoglobulins, mainly IgG, make up about 75% of the whey proteins. Other substances found in bovine colostrum normally include casein, lactoferrin, alpha-lactalbumin, beta-lactoglobulin, and the growth factors insulin-like growth factor-1 (IGF-1), insulin-like growth factor-2 (IGF-2), transforming growth factor beta (TGFbeta) and betacellulin (a member of the epidermal growth factor (EGF) family). In addition, bovine colostrum contains vitamins, minerals, lipids and lactose. Bovine colostrum may also contain colostrinin, also known as proline-rich polypeptide (PRP), a substance found in ovine (sheep) colostrum.

Bovine colostrum suitable for use according to the current invention is marketed in several forms. Colostrum can be in a full fat form and in a defatted skimmed form. A suitable form to apply in the present invention is colostrum in the form of a powder, in which form It is usually provided, but any other form can be used in this invention e.g. tablets, bars, liquids. When prepared by microfiltration the colostrum whey mainly comprises whey proteins and their associated immunoglobulins and the growth factors IGF-1, IGF-2, TGFbeta and betacellulin. Substances such as lactose, fats, casein and lactalbumin are typically significantly reduced in microfiltered bovine colostrum.

When producing colostrum it is important that the proteins are retained in their native conformation i.e. that they are in their undenatured, biologically active form. It is noted however that since colostrum contains many biologically active ingredients and activities, when considering the biological activity of proteins in their native confirmation, this has to be done in view of the desired effect.

The inventors now found that there exists an unexpected correlation between the amount of undenatured IgG and the biological effect of colostrum supplementation on the intestinal permeability. There are many different types of colostrum present on the market and most of them, although claiming a biological effect, do not have the desired effect on intestinal permeability (e.g. see FIGS. 1 and 2), but sometimes comprise a certain biological activity on other parameters.

### Quantification of Undenatured IgG in Colostrum Samples.

### Single Radial Immunodiffusion

Determination of undenatured IgG can be done on a routine basis by using the single radial immunodiffusion (SRID) technique. This simple and robust technique is the preferred method used for the quantification of undenatured IgG in colostrum samples according to the present invention. The method was developed for a precise quantification of soluble proteins by Mancini et al., (Mancini G, Carbonara A O, Heremans J M (1965). Immunological quantitation of antigens by single radial immunodiffusion. Immunochemistry, 2: 235-254).

A specific polyclonal antiserum raised against the protein to be quantified is incorporated in a buffered agar gel 1.5-2 mm thick. Circular wells are punched and then filled with a drop (2-15 μl) of the sample to be analyzed. The agar plate is then incubated in a moist box. When recognized by the antiserum, the proteins are precipitated in the agar during their diffusion. After complete diffusion, the diameters of the ring-shaped precipitates are measured. The surface of the precipitates is directly proportional to the protein concentration in the sample. Diffusion must be performed at constant temperature, 37° C. being optimal.

With an adequate (usually 18-24 h) diffusion time, a linear regression is obtained between the ring surface and the protein concentration. Standard curves are constructed by plotting the diameter of the precipitating ring versus the square root of the protein concentration.

### HPLC Affinity Chromatography Using a Protein G Column.

Another method for the quantification of undenatured IgG in colostrum is based on the difference in affinity of the undenatured IgG and denatured IgG to a protein G matrix.

Analyses were performed according to Journal of AOAC International 89 (5): 1249-1256, September-October 2006. Undenatured IgG is defined as the demi water soluble IgG in colostrum. Total IgG (incl. denatured) is defined as the IgG in colostrum, soluble in 0.28M NaCl. The affinity column employed is comprised of a matrix incorporating Protein G molecules which at neutral pH, bind specifically to the Fc-region of IgG molecules. This affinity is lost at pH 2.6.

Both SRID and HPLC techniques resulted in similar quantities of undenatured IgG, as is depicted in the table 1 below. In general the HPLC method resulted in a little lower value than the SRID method.

TABLE 1
Comparison of HPLC and SRID

| | |
|---|---|
| rehydration solution | demi water |

rehydration procedure

| | |
|---|---|
| step1 | 30 minutes ultrasonic treatment |
| step2 | stay overnight in the fridge (8 C.) |
| step3 | again 30 minutes ultrasonic treatment |
| step4 | filter through 0.45u filter |

HPLC eluent

| | |
|---|---|
| native IgG | binding buffer: PBS 1:10 with demi pH = 7.4 |
| | elution buffer: 50 mM Glycine pH = 2.6 |

Results
wt. % native IgG of total colostrum powder in 8 different colostrum
samples

| Colostrum sample | HPLC | SRID |
|---|---|---|
| 001 | 14.9 | 15.3 |
| 002 | 16.9 | |
| 003 | 8.3 | 8.7 |
| 004 | 7.3 | 7.7 |
| 005 | 6.7 | 7.3 |
| 006 | 7.1 | 7.7 |
| 007 | 6.5 | 7.2 |
| 008 | 7.2 | 7.2 |

### Dietary Fibers

Dietary fibers as used in this invention are typically resistant to digestion and absorption in the human small intestine with preferably a complete or partial fermentation in the large intestine. Preferably the present composition comprises at least one dietary fiber selected from the group consisting of galactooligosaccharides including trans galactooligosaccharides, inulin, fructooligosaccharides, xylooligosaccharides, palatinoseoligosaccharide, soybean oligosaccharide, gentiooligosaccharide, pectin, pectate, alginate, chondroitine, hyaluronic acids, heparine, heparane, bacterial carbohydrates, sialoglycans, fucoidan, fucooligosaccharides, carrageenan, xanthan gum, cellulose, polydextrose (PDX), guar gum, and/or hydrolysates thereof. All of these have beneficial prebiotic effects in the intestinal system. PDX is a non-digestible carbohydrate that has been synthesized from randomly cross-linked glucose and sorbitol.

Preferably the present composition comprises a dietary fiber selected from the group consisting of galactooligosaccharides, fructooligosaccharides, inulin and pectin degradation product. These dietary fibers have been shown to modulate the intestinal flora in such a way that positive effects can be expected on gut permeability and improve the positive effects of the present colostrum.

Preferably at least 10 g of a colostrum sample selected according to the invention, corresponding to about at least 2 g of undenatured IgG, is given to a person for the treatment and/or prevention of intestinal disorders. In certain cases it is preferred to increase the daily dose to at least 20 g colostrum per day, corresponding to about 4 g undenatured IgG. The daily dose can be given in 1 or more doses and in 1 or more doses forms (powder, liquid or bar). The use of different doses forms will increase the compliance which is important for the effectiveness of the product.

### EXAMPLES

### Intestinal Permeability

The bio-assay for the determination of altered intestinal permeability was performed by seeding CaCo2 cells on transwell inserts. CaCo2 monolayers were used after 3 weeks of confluency. Different colostrum samples were added in the apical compartment and cytokines in the basolateral compartment. To determine the permeability of the monolayers transepithelial resistance (TER; ohm/cm²) was measured by epithelial voltohmmeter and the apical-to-basolateral flux of 4 kDa FITC-dextran (FD4 pmol FD4/cm²/h) was measured after 72 hours. Different colostrum samples were tested for their effect on the cytokine induced increase of intestinal permeability.

Experiments have shown that not all colostrum batches are effective in preventing cytokine induced increase in intestinal permeability when compared to controls such as albumin, casein and whey and that only colostrum selected on the basis of its undenatured immunoglobulin G content higher than 19% based on the total weight of colostrum powder i.e. in this case higher than 25% based on the weight of total protein in colostrum powder, can prevent cytokine induced increase of intestinal permeability. Table 2 shows the percentage undenatured IgG in different colostrum samples.

TABLE 2
Percentage undenatured IgG in colostrum

| Colostrum | % undenatured IgG* |
|---|---|
| C1 | 29.0 |
| C2 | 33.3 |
| C3 | 36.5 |
| C4 | 37.5 |
| C5 | 20.8 |
| C6 | 19.9 |
| C7 | 24.4 |
| C8 | 21.6 |
| C9 | 20.7 |

*based on total protein weight of colostrum powder

FIG. 1 shows the percentage protection of colostrum against cytokine induced increase of intestinal permeability after 72 hours incubation determined by transepithelial resistance (1a colostrum samples containing >25 wt. % undenatured IgG per total protein content of colostrum, 1b colostrum samples containing <25 wt. % undenatured IgG per total protein content of colostrum, 1c controls: BSA, Casein and Whey). These results indicate that persons with diseases associated with cytokine induced intestinal disorders will have improved intestinal barrier function when using colostrum with at least 25 wt. % undenatured IgG per total protein content of colostrum.

FIG. 2 shows the percentage protection against cytokine induced increase of intestinal permeability resulting from treatment with colostrum. The percentage protection is shown after 72 hours incubation with inflammatory cytokines and was determined by FD4 flux (2a colostrum samples containing >25 wt. % undenatured IgG per total protein content of colostrum, 2b colostrum samples containing <wt. 25% undenatured IgG per total protein content of colostrum, 2c controls: BSA, Casein and Whey). These results indicate that only when using the correctly selected colostrum with at least 25 wt. % undenatured IgG per total protein content of colostrum, a person with diseases associated with cytokine induced intestinal disorders will have improved intestinal barrier function.

FIG. 3 shows the percentage protection of pure undenatured IgG and colostrum containing the same concentration of pure undenatured IgG against cytokine induced increase of intestinal permeability after 72 hours incubation determined by TER (3a) and FD4 flux (3b). Pure undenatured IgG alone is not effective in preventing cytokine induced increase in intestinal permeability.

### Compositions

### Powder Composition Comprising

| | |
|---|---|
| Colostrum | 10 g |
| Maltodextrin | 3 g |
| Vit/mineral mix | 0.5 g |
| Flavor orange | |

### Bar Composition Comprising

| | |
|---|---|
| Colostrum | 20 g |
| GOS + FOS | 15 g |
| Pectin hydrolysate | 5 g |
| Maltodextrin | 2 g |
| Flavor chocolate | |

### Nutritional Composition

| | |
|---|---|
| Colostrum | 20 g |
| Fat | 10 g |
| EPA/DHA | 2 g |
| Maltodextrin | 10 g |
| guar gum | 1 g |
| xanthan gum | 0.2 g |
| Fructooligosaccharides | 1 g |
| Vitamins | according to FSMP |

### REFERENCES

1. Pemet Pet al. Scand J Gastroenterol, 1999 34:29-34
2. Fantini Jet al. Journal of virology, 1992 66(1):580-585.
3. Mc Gowan I et al. J Acquir Immune Defic Syndr, 2004 37(2):1228-1236
4. Sharpstone G R et al. Aids, 1996 10(9):989-994
5. Reka S et al. Lymphokine Cytokine Res, 1994 13(6):391-398
6. Mc Gowan I et al. Aids, 1994 8(11):1569-1575
7. Kotler D P et al. Dig Dis Sci, 1993 38(6):1119-1127
8. Schmitz H et al. Aids, 2002 16(7):983-991
9. Stockman M et al. Ann N Y Acad Sci, 2000 915:293-303
10. Schmitz H et al. J Cell Sci, 1999 112(Pt1):137-146
11. Stockmann M et al. Aids, 1998 12(1):43-51.

1-10. (canceled)

11. A method for the treatment and/or prevention of an intestinal disorder in a patient, the method comprising administering to the patient a medicament comprising colostrum, wherein the colostrum comprises more than 25 weight percent undenatured immunoglobulin G based on the weight of total protein content of the colostrum.

12. The method according to claim 11, wherein the colostrum comprises between 25-40 weight percent undenatured immunoglobulin G based on the weight of total protein content of the colostrum.

13. The method according to claim 11, wherein the medicament comprises at least 2 g undenatured immunoglobulin G in a daily dose.

14. The method according to claim 11, wherein the patient is infected with HIV.

15. The method according to claim 11, wherein the intestinal disorder is an intestinal permeability disorder.

16. The method according to claim 11, wherein the intestinal disorder is inflammatory bowel disease, HIV infection and AIDS induced diarrhea, food allergy, nonsteroidal anti-inflammatory drugs induced increase in intestinal permeability, malabsorption, or a combination thereof.

17. The method according to claim 11, wherein the composition further comprises indigestible dietary fibers.

18. A composition comprising colostrum and indigestible dietary fibers, wherein the colostrum comprises more than 25 weight % undenatured immunoglobulin G based on the weight of total protein content of said colostrum.

19. The composition according to claim 18, wherein the colostrum comprises between 25-40 weight % undenatured immunoglobulin G the weight of total protein content of said colostrum.

20. The composition according to claim 18, wherein the indigestible dietary fiber is selected from the group consisting of galactooligosaccharides, transgalactooligosaccharides, inulin, fructooligosaccharides, xylo oligosaccharides, polydextrose, pectin, pectate, alginate, chondroitine, hyaluronic acids, heparine, heparane, bacterial carbohydrates, sialoglycans, fucoidan, fucooligosaccharides, and carrageenan and hydrolysates thereof.
